(19) United States
(12) Patent Application Publication
(10) Pub. No.: US 2010/0047193 A1
(43) Pub. Date: Feb. 25, 2010

(54) FORMULATIONS INCLUDING SELF TANNING, SUN PROTECTION, AND SHAVING COMPOSITIONS

(76) Inventor: Yoram Fishman, Los Angeles, CA (US)

Correspondence Address: KONRAD RAYNES & VICTOR, LLP, 315 S. BEVERLY DRIVE, # 210, BEVERLY HILLS, CA, 90212, US

(21) Appl. No.: 12/197,264

(22) Filed: Aug. 24, 2008

(51) Int. Cl. A61K 8/30; A61Q 17/04

(52) U.S. Cl. 424/59; 424/73

(57) ABSTRACT

Formulations and methods are described. One embodiment relates to a self tanning formulation including a skin coloring active that stains the skin by reaction therewith. The self tanning formulation also includes a component comprising polyhydroxystearic acid and isononyl isononanoate and ethylhexyl isononanoate and sodium cocamidopropyl PG-dimonium chloride phosphate and methyl perfluorobutyl ether and methyl perfluoroisobutyl ether. The formulation also includes a surfactant and a solvent. Other embodiments relate to sun protection compositions and shaving compositions. Other embodiments are described and claimed.

### BACKGROUND AND PROBLEMS WITH RELATED ART

For certain types of products that are applied to the body, (for example, self tanning products and suntan lotions), it is often difficult for the user to know if the product has been properly applied to the body. For instance, self tanning products typically use DHA (Dihydroxyacetone), which reacts with proteins in the skin and develops color about 2-6 hours after application. The DHA interaction with proteins in the skin produces a staining of the skin that typically lasts for 3-10 days. The color slowly fades as the skin cells slough off, and reapplication is needed to keep the skin tanned. As a result of the time it takes for the reaction to proceed and color the skin, the user will not know if the product was applied evenly enough to the skin to avoid streaking of the color, until it is too late to easily do anything about it.

To deal with the problem of ensuring even distribution of the self tanning product on the skin, certain manufacturers started adding artificial colors including pigments, dyes, and micas to the product, so the user could gauge if the product was effectively distributed on the skin by monitoring the presence of the artificial color. However, there are problems created by the use of the colorant in the product. One problem with such an approach is that the colorant added may be transferred to clothing, bed sheets, or whatever else the user comes into contact with. To avoid transmission of the colorant, the user must wait for the colorant to dry on the skin, which may take, for example, up to an hour. In addition, at some point after application of the product to the skin, the artificial colorant must be washed off the skin. If washed off too soon, all or some of the DHA may be removed.

Another problem with the use of colorants in the product is that the colorant used may cover the skin and/or deposit into the pores of the skin, and thus fill a site that would otherwise be filled with the DHA. In other words, the colorant tends to compete with the DHA for sites in or on the skin, thus lessening the effectiveness of the self tanning product because less is delivered to the appropriate location on the skin.

Products such as shaving creams and gels, in which it is desired to form a foam product on the skin, typically include a concentrate and a propellant housed in a pressurized container such as a metal aerosol can. The propellant is typically a flammable hydrocarbon such as propane or isobutane.

### DETAILED DESCRIPTION

Certain embodiments of the present invention relate to products and methods for forming and using products where one can gauge whether the product has been properly applied to the body. Such products may include, but are not limited to, formulations such as emulsions, suspensions, and solutions. Example of such products include, but are not limited to, self tanning products and sun protection products.

Certain embodiments relate to self tanning products. Self tanning products in accordance with certain embodiments include compositions comprising effective amounts of an active skin coloring ingredient, a solvent, a surfactant, and a component that under certain circumstances may act as a self foaming agent. An example of the component that may act as a self foaming agent is a material having an INCI (International Nomenclature of Cosmetic Ingredients) name Polyhydroxystearic Acid (and) Isononyl Isononanoate (and) Ethylhexyl Isononanoate (and) Sodium Cocamidopropyl PG-Dimonium Chloride Phosphate (and) Methyl Perfluorobutyl Ether (and) Methyl Perfluoroisobutyl Ether, available as PHOENOMULSE CE-1, from Phoenix Chemical, Inc. This component (available as PHOENOMULSE CE-1) may have one or more of the following functions, including: (i) a self-foaming agent, (ii) a spreading agent, and (iii) a tactile modifier. When applied to skin, after a period of time that is typically less than 10 seconds, the component will become activated due to body heat and dehydration and will self foam. However, if the component is adequately rubbed onto the skin, it will break down and will not foam.

The combination of ingredients listed above may form a self tanning composition which includes an indication mechanism to ensure that the product has been suitably applied and rubbed into the skin. In accordance with certain embodiments, the composition is applied to the skin and rubbed to apply the composition to the area desired to be tanned. Adequate rubbing in all desired areas is needed to ensure the formation of an even tan. After waiting a period of time (in certain embodiments, less than 10 seconds), areas needing additional rubbing will foam up on the skin. Areas that have been adequately rubbed into the skin will not foam up on the skin. The formation of the foam indicates that such an area needs to be further rubbed to properly apply the composition.

An active skin coloring ingredient which may be used in various self tanning embodiments is DHA or Dihydroxyacetone (INCI name). Lower concentrations of DHA produce lighter color formation, while higher concentrations produce darker color formation, resulting in the simulation of a suntan on the skin. Another ingredient which may be used either together with DHA or alone is Erythrulose (INCI name), which reacts in a similar way with the skin as does DHA. Certain embodiments may utilize DHA in an amount ranging from about 0.5 weight percent to about 10 weight percent, with certain preferred embodiments including about 4 weight percent to about 7 weight percent. Certain preferred embodiments may also include up to about 2 weight percent Erythrulose mixed with the DHA. Other materials and quantities may also be used.

A solvent is also used in various self tanning embodiments. The solvent may include one or more components therein, with water being preferred in certain embodiments. Generally, the quantity of water used as a solvent may depend on the various other ingredients used. The solvent may be present in certain embodiments in a range of about 10 to about 90 weight percent, with certain preferred embodiments including about 10 to about 60 weight percent. The exact quantity of solvent is also somewhat dependent on the form of the product. For example, a product in lotion form may in certain preferred embodiments include about 40 to about 65 weight percent water. A product in spray form may in certain preferred embodiments include about 30 to about 50 weight percent water. A product in cream or butter form may in certain preferred embodiments include from about 10 to about 30 weight percent water. Deionized water is generally preferred. Other suitable solvent materials may also be used.

A surfactant is also used in various self tanning embodiments. The surfactant component may include one or more surfactant materials. The surfactant component generally acts to lower the surface tension of the liquid mixture (for example, inhibits water and oil from separating into layers in the mixture) and allows for increased spreadability and formation of foam with the PHOENOMULSE CE-1 and body heat, if the mixture is not adequately rubbed onto the skin. Any suitable surfactant may be used, including anionic, cationic, nonionic, and amphoteric surfactants. Examples of surfactants include, but are not limited to, various sulfates, sulfonates, carboxylates, phosphates, ethoxylates, glycosides, amides, betaines, amione oxides, and amphoacetates. One example of a surfactant which may be used is Decyl Glucoside (INCI name), available as PLANTAREN 2000, from Cognis Corp. The surfactant may be present in certain self tanning embodiments in an amount of from about 1 to about 20 weight percent, with certain preferred embodiments including about 2 to about 12 weight percent.

The component that may act as a self foaming agent (an example of which is available as PHOENOMULSE CE-1, as noted above) may be present in certain self tanning embodiments in an amount of about 2 to about 30 weight percent, with certain preferred embodiments including about 10 to about 20 weight percent.

Certain embodiments also relate to sun protection products, which typically have a sun protection factor (SPF). Sun protection products in accordance with certain embodiments include compositions comprising effective amounts of an ultraviolet radiation protection agent, a solvent, a surfactant, and the component that under certain circumstances may act as a self foaming agent as described above in connection with self tanning embodiments. This component, as noted above for the self tanning embodiments, has the INCI name Polyhydroxystearic Acid (and) Isononyl Isononanoate (and) Ethylhexyl Isononanoate (and) Sodium Cocamidopropyl PG-Dimonium Chloride Phosphate (and) Methyl Perfluorobutyl Ether (and) Methyl Perfluoroisobutyl Ether, available as PHOENOMULSE CE-1, from Phoenix Chemical, Inc. Certain embodiments of sun protection products may include PHOENOMULSE CE-1 present in an amount of from about 2 to about 30 weight percent, with certain preferred embodiments including about 10 to about 20 weight percent.

Any suitable ultraviolet radiation protection materials such as UV absorbing materials and/or UV blocking materials may be used. Examples include, but are not limited to: Homosalate (INCI name), available as EUSOLEX HMS, from EMD Chemicals; Octyl Methoxycinnamate (INCI name), available as SOLAROM OMC, from Frutarom USA Inc.; Benzophenone-3 (INCI name), available as NEO HELIOPAN BB, from Symrise; Octyl Salicylate (INCI name), available as SOLAROM OS, from Frutarom USA Inc.; Avobenzone (INCI name) and Butylmethoxydibenzoylmethane (INCI name), available as PARSOL 1789 and SOLAROM BMBM, from Frutarom USA Inc.; and Titanium Dioxide (INCI name). The ultraviolet radiation protection materials may be present in certain embodiments in an amount of from about 1 to about 45 weight percent.

A solvent is also used in various sun protection embodiments. The solvent may include one or more components therein, with water being preferred in certain embodiments. Generally, the quantity of water used as a solvent may depend on the various other ingredients used. The solvent may be present in certain embodiments in a range of about 10 to about 90 weight percent, with certain preferred embodiments including about 10 to about 60 weight percent. The exact quantity of solvent is also somewhat dependent on the form of the product. For example, a product in lotion form may in certain preferred embodiments include about 15 to about 55 weight percent water. A product in spray form may in certain preferred embodiments include about 15 to about 70 weight percent water. A product in cream or butter form may in certain preferred embodiments include less water than spray and lotion embodiments. Deionized water is generally preferred. Other suitable solvent materials may also be used.

Various sun protection embodiments may also include a surfactant. The surfactant component may include one or more surfactant materials. The surfactant component generally acts to lower the surface tension of the liquid mixture (for example, inhibits water and oil from separating into layers in the mixture) and allows for increased spreadability. Any suitable surfactant may be used, including anionic, cationic, nonionic, and amphoteric surfactants. Examples of surfactants include, but are not limited to, various sulfates, sulfonates, carboxylates, phosphates, ethoxylates, glycosides, amides, betaines, amione oxides, and amphoacetates. One example of a surfactant which may be used is Decyl Glucoside (INCI name), available as PLANTAREN 2000, from Cognis Corp. In certain embodiments, the surfactant is present in an amount of from about 1 to about 20 weight percent, with certain preferred embodiments including about 2 to about 12 weight percent.

The combination of ingredients listed above may form a SPF composition which includes an indication mechanism to ensure that the product has been suitably applied to the skin. In accordance with certain embodiments, the SPF composition is applied to the skin and rubbed to apply the composition to the area desired to be protected from the sun. Adequate rubbing in all desired areas is needed to ensure that the SPF composition is either absorbed into or properly distributed on the skin to provide protection. After waiting a period of time (in certain embodiments, less than 10 seconds), areas needing additional rubbing will foam up on the skin. Areas that have been adequately rubbed into or onto the skin will not foam up.

When water is used as a solvent in various embodiments (self tanning compositions, sun protection compositions, and other compositions), the water also acts to provide a product with improved feel when applied to the body. Lower quantities of water than those specified in the preferred ranges may yield products that feel undesirably dry on the skin. In addition, when lower quantities of water are used, it may be more difficult to dissolve the various ingredients.

Various embodiments of both self tanning compositions and SPF compositions may also include various other ingredients, including, but not limited to, thickeners, humectants, skin conditioning agents, viscosity decreasing agents, colorants, preservatives, fragrance, humectants, film formers, pH adjusters, emollients, antioxidants, reducing agents, and other suitable additives. Certain additives may have more than one function. A variety of suitable chemicals may be used for each of the functions carried out by the additives.

Thickeners may include, but are not limited to, polymers such as PVM/MA Decadiene Crosspolymer (INCI name), available as STABILEZE QM, available from ISP Technologies. Humectants may include, but are not limited to: Glycereth-26 (INCI name), available as BOTANIMOIST G-26, from Botanigenics, Inc.; and Glycerin (INCI name), available as Glycerin 99.5%, from Norman, Fox & Co. A chelating agent may include, but is not limited to, Disodium EDTA (INCI name), available as VERSENE NA, from Univar USA. In addition, pH adjusters may include, but are not limited to: Sodium Hydroxide 50%, available from Ashland Chemicals; Lactic Acid (INCI name), from Univar USA; and Aminomethyl Propanol (INCI name), available as AMP Regular from Ashland Chemicals. Emollients may include, but are not limited to: Mineral Oil (INCI name), available as KAYDOL Mineral Oil, from Coast Chemicals; Petrolatum (INCI name), available as Petrolatum SNOW WHITE, from Penreco; Glyceryl Stearate (and) PEG-100 Stearate (INCI name), available as ENDIMULSE 165-V, from Coast Chemicals; C12-C15 Alkyl Benzoate (INCI name), available as LIPONATE NEB, from Lipo Chemicals; Cetearyl Ethylhexanoate (and) Isopropyl Myristate (INCI name), available as CRODAMOL CAP, from Croda; Propylene Glycol Isostearate (INCI name), available as EMEREST 2384; from Pacific Coast Chemicals; Glyceryl Stearate SE (INCI name), available as LIPO GMS-470, from Lipo Chemicals; Cyclopentasiloxane (INCI name), available as DOW CORNING 245, from Dow Corning; and Panthenol (INCI name), available as D-Panthenol from Seltzer Chemical Inc.

Emulsifying agents may include, but are not limited to: PEG-20 Sorbitan Cocoate (INCI name), available as RHEODOL TW-L120, from Coast Chemical; Cetearyl Ethylhexanoate (and) Isopropyl Myristate (INCI name), available as CRODAMOL CAP, from Croda; Propylene Glycol Isostearate (INCI name), available as EMEREST 2384; Beeswax (INCI name), available as BOTANIWAX BW-100, from Botagenics, Inc.; Sorbitan Sesquioleate (INCI name), available as LIPOSORB SQO, from Lipo Chemicals; and PPG-2-Isideceth-12 (INCI name), available as GENAPOL EP 1022, from Calriant. Emulsion stabilizers may include, but are not limited to, Hydroxypropyl Methylcellulose (INCI name), available as METHOCEL K4M, from Univar USA; C12-12 Alkyl Benzoate, available as ADOL 52, from Ashland Chemicals; Acrylates/C10-30 Alkyl Acrylate Crosspolymer, available as PEMULEN TR-1, from Noveon; Carbomer (INCI name), available as CARBOPOL 2984, from Noveon; Acrylates/C10-30 Alkyl Acrylate Crosspolymer, available as CARBOPOL ETD 2020, from Noveon; and Cetearyl Alcohol (and) Ceteareth-20 (INCI name), available as PROMULGEN D, from Lubrizol. Reducing agents may include, but are not limited to: Sodium Sulfite (INCI name), available from Universal Preser-A-Chem, Inc.; and Sodium Metabisulfite (INCI name), available from Esseco USA. Binder agents may include, but are not limited to: Ceresine Wax, available as Ceresine Wax 252, from Strahl & Pitsch; and Polyacrylamide (and) C13-14 Isoparaffin (and) Laureth-7 (INCI name), available as SEPIGEL 305, from Seppic. Viscosity decreasing agents include, but are not limited to, Ethoxydiglycol (INCI name), available as TRANSCUTOL CG, from Gattefosse. Film forming agents include, but are not limited to, Tricontanyl PVP (INCI name), available as GANEX WP660, from ISP Technologies.

Skin conditioning agents may include, but are not limited to: Shea Butter (INCI name), available from Desert Whale Jojoba Co.; Sunflower Seed Oil (INCI name), available from Desert Whale Jojoba Co.; Black Walnut Extract (INCI name), available from Vegetech; Dimethicone (INCI name), available as DOW CORNING 200 Fluid, available from Dow Corning; Witch Hazel Extract (INCI name), available from Vegetech; Water (and) Propylene Glycol (and) Walnut Extract (INCI name), available as CRODAROM NUT A, from Croda; Butylene Glycol (INCI name), available from Ashland Chemicals; and Saccharum Offic. (sugar cane) Extract (and) Citrus Medica Limonum (lemon) Extract (and) Pyrus Malus (apple) Extract (and) Camellia Sinensis Leaf Extract (INCI name), available as MFA COMPLEX, from Barnet Products Corp.

Antioxidants may include, but are not limited to: Tocopheryl Acetate (INCI name), available as dl-Alpha Tocopheryl Acetate, from Seltzer Chemical, Inc.; Sodium Sulfite (INCI name), available from Universal Preser-A-Chem, Inc.; Sodium Metabisulfite (INCI name), available from Esseco USA; and BHT (INCI name), available as BHT or butylated hydroxytoluene, from Univar USA. Preservatives, may include, but are not limited to: Phenoxyethanol (and) Caprylyl Glycol (and) Ethylhexylglycerin (and) Hexylene Glycol (INCI name), available as BOTANISTAT PF-64, from Botanigenics, Inc.; 2-Phenoxyethanol (INCI name), available as EMERESSENCE 1160, from Pacific Coast Chemicals; and Caprylyl Glycol (and) Phenoxyethanol (and) Hexylene Glycol (INCI name), available as DIOCIDE, from DIOW Products, Inc. Fragrance (Parfum) (INCI name) may also be used to impart or mask odor. Additional suitable additives (including, but not limited to, vitamins and minerals) may also be included in various embodiments.

Examples of self tanning butter, self tanning lotion, and self tanning spray compositions according to certain embodiments are set forth in Table 1 below. The self tanning butter and lotion embodiments set forth in Table 1 are formed as emulsions. The self tanning spray embodiment set forth in Table 1 is formed as a solution.

Examples of SPF compositions in spray and lotion form are set forth in Table 2 below. Other forms, including, but not limited to, a butter form, are also possible.

TABLE 1
Examples of self tanning butter, self tanning lotions, and self tanning spray
compositions.

| Ingredient | Butter | Lotion A | Lotion B | Spray |
|---|---|---|---|---|
| Purified Water | 21.850 wt % | 53.960 wt % | 49.600 wt % | 41.400 wt % |
| STABILEZE QM | 1.000 | 0.500 | — | — |
| BOTANISTAT PF-64 | 1.000 | 1.000 | 1.000 | — |
| Sodium Hydroxide 50% | 0.200 | 0.300 | — | — |
| KAYDOL Mineral Oil | 15.000 | — | — | — |
| Ceresine Wax 252 | 7.500 | — | — | — |
| Petrolatum SNOW WHITE | 7.000 | — | — | — |
| BOTANIWAX BW-100 | 6.750 | — | — | — |
| LIPOSORB SQO | 6.000 | — | — | — |
| GENAPOL EP 1022 | 3.500 | — | — | — |
| Fragrance | 1.000 | 0.500 | 0.500 | 0.500 |
| Black Walnut Extract | 0.200 | 0.100 | — | 0.100 |
| Erythrulose | 0.500 | 0.500 | — | 0.500 |
| Dihydroxyacetone | 6.000 | 6.000 | 5.000 | 6.000 |
| Sodium Sulfite | 0.200 | — | — | — |
| Sodium Metabisulfite | 0.300 | 0.050 | 0.100 | — |
| PLANTAREN 2000 | 7.000 | 7.000 | 7.000 | 7.000 |
| PHOENOMULSE CE-1 | 15.000 | 15.000 | 15.000 | 15.000 |
| BOTANIMOIST G-26 | — | 2.000 | — | — |
| VERSENE NA | — | 0.050 | — | — |
| PROMULGEN D | — | 1.500 | — | — |
| ENDIMULSE 165-V | — | 1.000 | 4.500 | — |
| LIPO GMS-470 | — | 1.500 | — | — |
| Shea Butter | — | 2.000 | — | — |
| Sunflower Oil | — | 3.000 | — | — |
| BHT | — | 0.050 | — | — |
| DOW CORNING 245 | — | 2.000 | 8.200 | — |
| D-Panthenol | — | 0.100 | — | — |
| dl-Alpha Tocopherol Acetate | — | 0.100 | — | — |
| SEPIGEL 305 | — | 1.200 | 2.000 | — |
| Lactic Acid | — | 0.590 | — | — |
| Glycerin 99.5% | — | — | 2.000 | — |
| CRODAMOL CAP | — | — | 2.500 | — |
| ADOL 52 | — | — | 0.800 | — |
| LIPONATE NEB | — | — | 1.600 | — |
| CRODAROM NUT A | — | — | 0.100 | — |
| MFA Complex | — | — | 0.100 | — |
| Witch Hazel Extract | — | — | — | 10.000 |
| Butylene Glycol | — | — | — | 4.000 |
| TRANSCUTOL CG | — | — | — | 15.000 |
| EMERESSENCE 1160 | — | — | — | 0.500 |
| | 100.000 | 100.000 | 100.000 | 100.000 |

TABLE 2
Examples of SPF-30 compositions.

| Ingredient | Lotion | | Spray | |
|---|---|---|---|---|
| Purified Water | 30.820 | wt % | 33.430 | wt % |
| CARBOPOL 2984 | 0.120 | | — | |
| VERSENE NA | 0.050 | | — | |
| VERSENE 220 | — | | 0.100 | |
| METHOCEL K4M | 0.100 | | — | |
| EUSOLEX HMS | 13.000 | | 15.000 | |
| SOLAROM OMC | 7.500 | | 7.500 | |
| NEO HELIOPAN BB | 6.000 | | 6.000 | |
| SOLAROM OS | 5.000 | | 5.000 | |
| ENDIMULSE 165-V | 3.000 | | — | |
| PARSOL 1789/SOLAROM BMBM | 2.000 | | — | |
| LIPONATE NEB | 1.000 | | 5.000 | |
| PEMULEN TR-1 | 0.300 | | — | |
| AMP REGULAR | 0.360 | | — | |
| EMEREST 2384 | 5.000 | | — | |
| DOW CORNING 200 | 1.000 | | 0.250 | |
| DIOCIDE | 1.000 | | — | |
| Fragrance | 1.750 | | 2.000 | |
| PLANTAREN 2000 | 7.000 | | 7.000 | |
| PHOENOMULSE CE-1 | 15.000 | | 15.000 | |
| CARBOPOL ETD 2020 | — | | 0.120 | |
| GANEX WP660 | — | | 0.500 | |
| BOTANISTAT PF-64 | — | | 1.000 | |
| Triethanolamine 85% | — | | 0.100 | |
| RHEODOL TW-L120 | — | | 2.000 | |
| | 100.000 | | 100.000 | |

Certain embodiments also relate to methods for forming products, for example, self tanning formulations having the ingredients such as listed in Table 1, and sun protection products having the ingredients listed in Table 2.

In one embodiment for the preparation of a self tanning butter having the ingredients listed in Table 1, about 90 weight percent of the purified water is metered into a main mixing tank. The STABILEZE QM is added and mixed well at 70-75° C., until uniform. At 70-75° C., the Sodium Hydroxide 50% is mixed into the batch. In a separate container, ingredients including KAYDOL MINERAL OIL, CERESINE WAX 252, PETROLATUM SNOW WHITE, BOTANIWAX BW-100, LIPOSORB SQO, and GENAPOL EP 1022 are added and mixed well at 70-75° C., until all solids are dissolved. While at 70-75° C., the contents of the separate container are mixed into the contents in the main mixing tank and mixed for 15-30 minutes to emulsify. The batch is then cooled. At 40-45° C., the BOTANISTAT PF-64, the Fragrance, the Black Walnut Extract, and the Erythrulose are mixed into the batch. The batch may then continue to be cooled.

In a separate container, the remaining purified water (about 10 weight percent), the DHA, and the Sodium Sulfite are mixed at a temperature of 40-42° C., the cooled below 40° C., at which time Sodium Metabisulfite is mixed in. Then the contents of the separate container are added to the main batch. In the temperature range of 35-40° C., the PLANTAREN 200 is added and the batch well mixed. The batch is cooled further. At 12-18° C., the PHOENOMULSE CE-1 is added to the batch and mixed well. The batch may then be transferred into suitable containers.

In another embodiment, a self tanning lotion having the ingredients listed in Table 1 as Lotion A is formed. About 90 weight percent of the purified water is added to a main mixing tank. The STABILEZE QM is added and mixed until completely hydrated. The Sodium Hydroxide 50% is added to the tank, followed by the BOTANIMOIST G-26 and the VERSENE NA. The batch in the main tank is heated to heated to 70-75° C. and mixed until substantially uniform. In a separate container, the PROMULGEN D, ENDIMULSE 165-V, LIPO GMS-470, Shea Butter, Sunflower Oil, and BHT are heated to 70-75° C. and mixed until all solids are dissolved. With each at 70-75° C., the components from the separate container are added to those in the main tank and mixed for about 15-30 minutes to emulsify. Then the batch is cooled. At 60-65° C., the DOW CORNING 245 Fluid is added to the batch and mixed well. In a separate container, the DHA and Erythrulose are mixed with the remaining amount of the purified water (about 10 weight percent) and the Sodium Metabisulfite, then added to the batch at 40-45° C. and mixed well.

Then additional ingredients including the Black Walnut Extract, D-Panthenol, DL-Alpha Tocopheryl Acetate, BOTANISTAT PF-64, SEPIGEL 305, Fragrance, and Lactic Acid are added to the batch and mixed well. The PLANTAREN 2000 is then added to the batch and mixed well. The batch is then cooled. At 12-18° C., the PHOENOMULSE CE-1 is added to the batch and mixed well. The product may then be transferred into suitable containers.

In another embodiment, a self tanning lotion having the ingredients listed in Table 1 as Lotion B is formed. About 90 weight percent of the purified water is added to a main mixing tank. The Glycerin 99.5% is added and mixed at 70-75° C. until uniform. In a separate container, the ENDIMULSE 165-V, CRODAMOL CAP, ADOL 52, and LIPONATE NEB are mixed at 70-75° C. until all solids are dissolved. The contents of the separate container are added to the contents in the main mixing tank at 70-75° C. and mixed for approximately 15-30 minutes to emulsify the batch. The batch is cooled and at 60-65° C., the DOW CORNING 245 Fluid is added to the batch and mixed well. At 55-60° C., the BOTANISTAT PF-64 and the SEPIGEL 305 are added to the batch and mixed well. At 45-50° C., the CRODAROM NUT A and the Fragrance are added to the batch and mixed well.

In a separate container, the DHA, about 10 weight percent of the purified water, and the Sodium Metabisulfite are premixed and then added to the batch and mixed well. The MFA Complex and PLANTAREN 2000 are then added to the batch and mixed well. The batch is then cooled. At 12-18° C., the PHOENOMULSE CE-1 is added to the batch and mixed well. The product may then be transferred into suitable containers.

In another embodiment a self tanning spray having the ingredients listed in Table 1 is formed. In a main mixing tank, the purified water is added. Then the following ingredients are added one at a time, with the batch mixed well after each is added: Witch Hazel Extract, Butylene Glycol, Erythrulose, Black Walnut Extract, TRANSCUTOL CG, DHA, and EMERESSENCE 1160. Then, the Fragrance is added, followed by the PLANTAREN 2000. The batch is mixed well and then cooled. At 12-18° C., the PHOENOMULSE CE-1 is added to the batch and mixed well. The product may then be transferred into suitable containers.

In another embodiment, a sun protection lotion having SPF-30 and containing the ingredients listed in Table 2 is formed. In a main mixing tank, the purified water is metered into the tank. The CARBOPOL 2984 is then slowly sprinkled in and mixed until hydrated. The batch is then heated to 75-80° C. and then the VERSENE NA and METHOCEL K4M are added and the batch mixed until substantially uniform. While maintaining the batch temperature at 75-80° C., the following ingredients are added in the order listed, mixing well after each is added: EUSOLEX HMS, SOLAROM OMC, NEO HELIOPAN BB, SOLAROM OS, ENDIMULSE 165-V, and PARSOL 1789/SOLAROM BMBM.

In a separate container, the LIPONATE NEB and PEMULEN TR-1 are mixed at 75-80° C. and then added to the batch in the main mixing tank and mixed at 75-80° C. Then the AMP REGULAR is added and mixed until the batch is emulsified. Then the EMEREST 2384, the DOW CORNING 200 Fluid, the DIOCIDE, and the Fragrance are mixed into the batch until substantially uniform. Then the PLANTAREN 2000 is mixed into the batch and then the batch is cooled. At 12-18° C., the PHOENOMULSE CE-1 is added to the batch and mixed well. The product may then be transferred into suitable containers.

In still another embodiment, a sun protection spray having SPF-30 and containing the ingredients listed in Table 2 is formed. In a main mixing tank, the purified water is metered into the tank. The VERSENE 220 and CARBOPOL ETD 2020 are then added. The batch is heated to 75-80° C. and mixed until substantially uniform. In a separate container, the LIPONATE NEB, GANEX WP660, SOLAROM OMC, EUSOLEX HMS, NEO HELIOPAN BB, and SOLAROM OS are added and heated to 75-80° C. and mixed until all solids are dissolved. At 75-80° C., the mixed separate container components are mixed into the batch in the main mixing tank and mixed until uniform. The Triethanolamine 99% and RHEODOL TW-L120 are added to the batch and mixed using a homogenizer until the batch is emulsified. The batch is then cooled and at 35-40° C., the BOTANISTAT PF-64, the Fragrance and DOW CORNING 200 Fluid are added to the batch and mixed well. Next, the PLANTAREN 2000 is added to the batch and mixed well. The batch is cooled and at 12-18° C., the PHOENOMULSE CE-1 is added to the batch and mixed well. The product may then be transferred into suitable containers.

A variety of modifications may be made to the processes described above. For example, in certain embodiments, the order of adding certain ingredients may be modified. Also, in certain embodiments, some of the temperatures of the various processing operations may be modified.

It should be noted that the classification of ingredients by function is not always exact and may be somewhat arbitrary. For example, Hydroxypropyl Methylcellulose may be classified as an emulsion stabilizer and as a film former. Ethoxydiglycol may be characterized as a viscosity decreasing agent and as a solvent. Tricontanyl PVP may be characterized as a viscosity increasing agent and a film former. CARBOPOL ETD 2020 may be characterized as an emulsion stabilizer and thickener. Consequently, for purposes of the descriptions herein, the components are characterized according to what is believed to be a useful role (not necessarily the only one) in providing the desired properties as used in compositions described herein.

Other embodiments are also possible, their specific features depending upon the particular application. For example, embodiments of the present invention may be applicable to a variety of types of products for application to various parts of the body. For instance, embodiments may also be applicable to other types of compositions that are applied and rubbed into the skin, where an even application is desired. Such embodiments may include medications and/or skin treatment ingredients. Such embodiments may include a combination of ingredients including the active ingredient or ingredients to treat the desired condition, a solvent, a surfactant, and a component that results in the composition foaming on the skin if it has not been adequately applied to the skin, such as the Polyhydroxystearic Acid (and) Isononyl Isononanoate (and) Ethylhexyl Isononanoate (and) Sodium Cocamidopropyl PG-Dimonium Chloride Phosphate (and) Methyl Perfluorobutyl Ether (and) Methyl Perfluoroisobutyl Ether component described above, available as PHOENOMULSE CE-1. Such embodiments may include, but are not limited to, anti-itch products, antibiotic products, anti-fungal products, poison ivy treatment products, psoriasis treatment products, pain relief products, and acne treatment products. For instance, active ingredients in an anti-itch embodiment may include, but are not limited to, Benzyl Alcohol (INCI name) and/or Hydrocortisone Acetate (INCI name). Active ingredients in an antibiotic treatment embodiment may include, but are not limited to, Bacitracin Zinc and/or Polymyxin-B Sulfate. Active ingredients in an anti-fungal embodiment may include, but are not limited to, Tolnaftate (INCI name). Active ingredients in a poison ivy treatment embodiment may include, but are not limited to, Pramoxine Hydrochloride. Active ingredients in a psoriasis treatment embodiments may include, but are not limited to, Coal Tar. Active ingredients in a pain relief embodiment may include, but are not limited to, Bacitracin Pramoxine HCl. Active ingredients in an acne treatment gel or other topical gel may include, but are not limited to, Sulfur 1x.

Still other embodiments relate to shaving formulations and the like, including, but not limited to, shaving creams and shaving gels.

Shaving creams in accordance with certain embodiments include compositions comprising effective amounts of a surfactant, an oil phase, a neutralizer, a solvent, and a component that may act as a self foaming agent. This component may have the INCI name Polyhydroxystearic Acid (and) Isononyl Isononanoate (and) Ethylhexyl Isononanoate (and) Sodium Cocamidopropyl PG-Dimonium Chloride Phosphate (and) Methyl Perfluorobutyl Ether (and) Methyl Perfluoroisobutyl Ether, available as PHOENOMULSE CE-1, from Phoenix Chemical, Inc.

A surfactant that may be used in shaving cream formulations includes, but it not limited to, Decyl Glucoside (INCI name), available as PLANTAREN 2000, from Cognis Corp. Certain embodiments include about 1 to about 25 weight percent surfactant, with certain preferred embodiments including about 2 to about 10 weight percent. A preferred solvent is water. Certain shaving cream embodiments may include about 10 to about 90 weight percent solvent, with certain preferred embodiments including about 25 to abut 65 weight percent. The oil phase may include, for example, one or more fatty acids and/or oils such as almond oil. Certain shaving cream embodiments include about 1 to about 50 weight percent oil phase, with certain preferred embodiments including about 15 to about 40 weight percent oil phase. The neutralizer may be an alkaline material including, but not limited to, potassium hydroxide and/or triethanolamine, and may be present in certain embodiments at about 0.5 to about 15 weight percent, with certain preferred embodiments including about 2 to about 8 weight percent. The neutralizer reacts with the fatty acids to form a soap. Certain shaving cream embodiments may include about 2 to about 30 weight percent of the PHOENOMULSE CE-1, with certain preferred embodiments including about 5 to about 15 weight percent.

Shaving gels in accordance with certain embodiments include compositions comprising effective amounts of a surfactant, a thickener, a solvent, and the component that may act as a self foaming agent as described above (available as PHOENOMULSE CE-1). A surfactant that may be used in shaving gel formulations includes, but is not limited to, Sodium Cocoyl Isethionate (INCI name), available as HOSTAPON SCI-85G, from Clariant. Certain shaving gel embodiments include about 1 to about 30 weight percent surfactant, with certain preferred embodiments including about 2 to about 20 weight percent. A thickener includes, but is not limited to, Hydroxypropyl Guar (INCI name), available as JAGUAR HP-60, from Chemtec. Certain shaving gel embodiments include about 0.1 to about 10 weight percent thickener. A preferred solvent is water. Certain shaving gel embodiments may include about 10 to about 90 weight percent solvent, with certain preferred embodiments including about 40 to about 80 weight percent solvent. Certain shaving gel embodiments may include about 2 to about 30 weight percent of the PHOENOMULSE CE-1, with certain preferred embodiments including about 5 to about 15 weight percent. Shaving gel embodiments and shaving cream embodiments may be formed as emulsions.

Other ingredients may be substituted where suitable for the shaving cream and shaving gel ingredients set forth above. In addition, shaving cream and shaving gel embodiments may include various additional ingredients to those listed above. For example, shaving cream embodiments may include a thickener, and shaving gel embodiments may include fatty acids and oils. Additional ingredients may include, but not limited to, fatty acids, oils, thickeners, humectants, chelating agents, skin conditioning agents, viscosity decreasing agents, colorants, preservatives, fragrance, pH adjusters, emollients, antioxidants, reducing agents, botanical extracts and other suitable additives. Certain additives may have more than one function. In addition, the additives are not limited to the specific ingredients set forth herein.

Examples of certain specific ingredients that may be used in shave cream and/or shave gel embodiments are set forth below. Other suitable materials may be used in place of or in addition to the ingredients listed. As noted earlier, some ingredients may have more than one function and there may be overlapping functional classifications. Various of the ingredients such as acids and neutralizers react when mixed to form soaps. An example of a humectant is Glycerin (INCI name), available from Westco. An example of a chelating agent is Tetrasodium EDTA (INCI name), available as VERSENE 220, available from Univar. Examples of fatty acids include Myristic Acid (INCI name), available from Wilchem; Stearic Acid (INCI name), available from Ashland; and Cocus Nucifera (Coconut) Acid (INCI name), available as GEROPON AC-78NP, from Actichem. Examples of emulsifying agents include Sodium Isostearyl Lactylate (INCI name), available as PATIONIC ISL, from Rita Corp.; and Glyceryl Stearate SE, available as CERASYN Q, from ISP Tech.

Skin conditioning agents include Butylene Glycol, available from Ashland; and Prunus Amygdalus Dulcis (Sweet Almond) Oil (INCI name), available as Almond Oil, from Lipo Chemicals. An example of a skin and hair conditioning agent is Panthenol (INCI name), available as DL-Panthenol from Seltzer Chemicals. An example of a skin protectant is Allantoin (INCI name), available from ISP Tech. An example of an emollient is Sunflower Seed Oil Glycerides (INCI name), available as MYVEROL 18-92K, from Univar. An example of a botanical extract is Aloe Barbadensis Leaf Juice, (INCI name), available as BOTANIVERA 104, from Botanigenics, Inc. An example of an antioxidant is Tocopheryl Acetate (INCI name), available as dl-Alpha Tocopheryl Acetate, from Seltzer Chemicals. An example of a preservative is Phenoxyethanol (and) Caprylyl Glycol (and) Ethylhexylglycerin (and) Hexylene Glycol (INCI name), available as BOTANISTAT PF-64, from Botanigenics, Inc. Fragrance (Parfum) may also be added to the compositions to mask or impart an odor. Other suitable additives (including, but not limited to, vitamins and minerals) may also be included in various embodiments.

Examples of a shaving cream composition and a shaving gel composition according to certain embodiments are set forth in Table 3 below.

TABLE 3
Examples of shaving cream and shaving gel compositions.

| Ingredient | Shave Gel | | Shave Cream | |
|---|---|---|---|---|
| Purified Water | 60.170 | wt % | 44.970 | wt % |
| JAGUAR HP-60 | 0.360 | | 0.100 | |
| Glycerine | 7.800 | | 3.600 | |
| VERSENE 220 | 0.100 | | — | |
| Allantoin | 0.050 | | — | |
| HOSTAPON SCI-85G | 10.800 | | — | |
| PATIONIC ISL | 1.100 | | — | |
| Myristic Acid | 7.200 | | 9.000 | |
| CERASYN Q | 1.800 | | | |
| PLANTAREN 2000 | — | | 5.000 | |
| MYVERSOL 18-92K | 1.000 | | — | |
| dl-Alpha Tocopheryl Acetate | 0.010 | | 0.010 | |
| DL-Panthenol | 0.010 | | 0.010 | |
| Fragrance | 0.600 | | 1.300 | |
| PHOENOMULSE CE-1 | 8.000 | | 10.000 | |
| Stearic Acid | — | | 15.200 | |
| Potassium Hydroxide | — | | 4.000 | |
| GEROPON AC-78NP | — | | 2.700 | |
| Triethanolamine | — | | 1.100 | |
| Butylene Glycol | — | | 1.000 | |
| Almond Oil | — | | 1.000 | |
| BOTANISTAT PF-64 | 1.000 | | 1.000 | |
| BOTANIVERA 104 | — | | 0.010 | |
| | 100.000 | | 100.000 | |

Certain embodiments also relate to methods for forming shaving compositions, for example, formulations having the ingredients listed in Table 3.

In one embodiment for the preparation of shaving gel having the ingredients listed in Table 3, the purified water is metered into a main mixing tank. With high speed agitation, the JAGUAR HP-60 is sprinkled in and mixed until fully hydrated. Then the Glycerine and Allantoin are added and the batch heated to 70-75° C. At 70-75° C., the HOSTAPON SCI-85G and the PATIONIC ISL are added and mixed until all solids are dissolved and the batch is uniform. In a separate container, the Myristic Acid, CERASYN Q, and MYVEROL 18-92K are mixed and heated to 70-75° C. With the contents of both containers at 70-75° C., the contents of the separate container are added to the batch in the main mixing tank and mixed well. The batch is then cooled to 45-50° C. and the BOTANISTAT PF-64 is added and mixed well. The batch is then cooled to 40-45° C. The batch is then cooled and at 35-40° C., the DL-Alpha Tocopheryl Acetate, DL-Panthenol, and Fragrance oil are added and mixed well. The batch is cooled at 12-18° C., the PHOENOMULSE CE-1 is added and mixed well. The batch may then be transferred to suitable containers.

In another embodiment of a shaving gel composition, Menthol (INCI name), available as Menthol Crystals, from Fragrance West, may be added. In this embodiment, the composition is the same as that of the Shave Gel listed in Table 3, except that 0.500 weight percent of Menthol Crystals are added (with 0.500 weight percent less of the Purified Water). The Menthol crystals are mixed with the Fragrance oil then added to the batch at the same time the Fragrance oil is added as described above.

In an embodiment for the preparation of shaving cream having the ingredients listed in Table 3, the purified water is metered into a main mixing tank. The Potassium Hydroxide, Triethanolamine, Glycerine, and PLANTAREN 2000 are added and mixed well. The batch is heated to 80-85° C. In a separate container, the JAGUAR HP-60 and Butylene Glycol are mixed and then added to the batch in the main container. In another separate container, the Stearic Acid, the Myristic Acid, the GERAPON AC-78NP, and the Almond Oil are mixed well and heated to 80-85° C. and then slowly mixed with the contents of the main mixing tank at 80-85° C. The batch is then cooled to 40-45° C. At 40-45° C., the BOTANISTAT PF-64, DL-Alpha Tocopheryl Acetate, DL-Panthenol, BOTANIVERA 104, and Fragrance oil are added and mixed well. The batch is cooled and at 12-18° C., the PHOENOMULSE CE-1 is added and mixed well. The batch may then be transferred to suitable containers.

It should be appreciated that the shaving cream and shaving gel embodiments described above are both water soluble and do not require a propellant phase such as in conventional aerosol shaving creams and gels, which requires a pressurized can (such as an aluminum or other metal can) and is flammable. Thus, embodiments permit a simpler approach to packaging than conventional aerosol shaving creams and shaving gels. For example, various embodiments described herein may be housed in a variety of containers including, but not limited to, a non-pressurized bottle formed from materials including, but not limited to, polyethylene or polypropylene.

The component that is described as one that may act as a self foaming agent, available as PHOENOMULSE CE-1, is used in various embodiments described herein. Various self tanning and sun protection embodiments utilize the PHOENOMULSE CE-1 as an indicator, so that when foam forms, the user knows to further rub the composition to distribute an active component (for example, skin colorant or UV protector) into or onto the skin. If the self tanning or sun protection composition is rubbed sufficiently into or onto the skin after application, the composition will not self foam because the repeated rubbing action breaks down the structure of the PHOENOMULSE so that the composition it will not self foam. For various shaving cream and shaving gel embodiments, the PHOENOMULSE CE-1 is used to provide a foaming composition that is generally not rubbed onto or into the skin to the same extent as a self tanning composition or sun protection composition and as a result, the composition will self foam. Once the foam is formed, the user is ready to then shave the hair on the skin.

The exact form of embodiments may change depending on factors such as the desired consistency and the selected ingredients. Since there may be many modifications without departing from the scope of the invention, the examples set forth herein are not intended to limit the invention but to illustrate certain aspects of the invention more clearly.

It will, of course, be understood that modifications of embodiments of the present invention, in its various aspects, will be apparent to those skilled in the art. The scope of the invention should not be limited by the particular embodiments described above. In addition, the terms “including”, “comprising”, “having” and variations thereof mean “including, but not limited to”, unless expressly specified otherwise. A listing of items does not imply that any or all of the items are mutually exclusive, unless expressly specified otherwise. The terms “a”, “an” and “the” mean “one or more”, unless expressly specified otherwise.

What is claimed is:

1. A self tanning formulation comprising:
a skin coloring active that stains the skin by reaction therewith;
a surfactant;
a component comprising polyhydroxystearic acid and isononyl isononanoate and ethylhexyl isononanoate and sodium cocamidopropyl PG-dimonium chloride phosphate and methyl perfluorobutyl ether and methyl perfluoroisobutyl ether; and
a solvent.

2. The formulation of claim 1, further comprising a thickener.

3. The formulation of claim 1, wherein the solvent comprises water.

4. The formulation of claim 1, wherein the skin coloring active comprises at least one material selected from the group consisting of dihydroxyacetone and erythrulose.

5. The formulation of claim 1, wherein the surfactant comprises at least one surfactant selected from the group consisting of anionic, cationic, nonionic, and amphoteric surfactants.

6. The formulation of claim 1, wherein the surfactant comprises decyl glucoside.

7. The formulation of claim 1, comprising:
10-90 weight percent of the solvent;
0.5-10 weight percent of the skin coloring active that stains the skin by reaction therewith;
1-20 weight percent of the surfactant; and
2-30 weight percent of the component comprising polyhydroxystearic acid and isononyl isononanoate and ethylhexyl isononanoate and sodium cocamidopropyl PG-dimonium chloride phosphate and methyl perfluorobutyl ether and methyl perfluoroisobutyl ether.

8. A sun protection formulation comprising:
an ultraviolet radiation protection agent;
a surfactant;
a component comprising polyhydroxystearic acid and isononyl isononanoate and ethylhexyl isononanoate and sodium cocamidopropyl PG-dimonium chloride phosphate and methyl perfluorobutyl ether and methyl perfluoroisobutyl ether; and
a solvent.

9. The formulation of claim 8, wherein the solvent comprises water.

10. The formulation of claim 8, wherein the surfactant comprises decyl glucoside.

11. The formulation of claim 8, wherein the surfactant comprises at least one surfactant selected from the group consisting of anionic, cationic, nonionic, and amphoteric.

12. The formulation of claim 8, comprising:
10-90 weight percent of the solvent;
1-45 weight percent of the ultraviolet radiation protection agent;
1-20 weight percent of the surfactant; and
2-30 weight percent of the component comprising polyhydroxystearic acid and isononyl isononanoate and ethylhexyl isononanoate and sodium cocamidopropyl PG-dimonium chloride phosphate and methyl perfluorobutyl ether and methyl perfluoroisobutyl ether.

13. A method of applying a formulation to skin, comprising:
providing a formulation comprising: (i) a surfactant, (ii) a component comprising polyhydroxystearic acid and isononyl isononanoate and ethylhexyl isononanoate and sodium cocamidopropyl PG-dimonium chloride phosphate and methyl perfluorobutyl ether and methyl perfluoroisobutyl ether, and (iii) a solvent;
applying the formulation to the skin;
waiting a period of time after the application of the formulation to the skin;
determining whether any foam forms on the skin after the period of time and if the foam forms on the skin, rubbing the foam on the skin.

14. The method of claim 13, wherein the formulation further comprises at least one of (i) a skin coloring active that stains the skin by reaction therewith and (ii) an ultraviolet radiation protection agent.

15. A shaving formulation comprising:
an oil phase;
a surfactant;
a neutralizer;
a component comprising polyhydroxystearic acid and isononyl isononanoate and ethylhexyl isononanoate and sodium cocamidopropyl PG-dimonium chloride phosphate and methyl perfluorobutyl ether and methyl perfluoroisobutyl ether; and
a solvent.

16. The formulation of claim 15, wherein the solvent comprises water.

17. The shaving formulation of claim 15, comprising:
1 to 50 weight percent oil phase;
1 to 25 weight percent surfactant
0.5 to 15 weight percent neutralizer;
2 to 30 weight percent of the component comprising polyhydroxystearic acid and isononyl isononanoate and ethylhexyl isononanoate and sodium cocamidopropyl PG-dimonium chloride phosphate and methyl perfluorobutyl ether and methyl perfluoroisobutyl ether; and
10 to 90 weight percent of the solvent, the solvent comprising water.

18. A shaving formulation comprising:
a surfactant;
a thickener;
a component comprising polyhydroxystearic acid and isononyl isononanoate and ethylhexyl isononanoate and sodium cocamidopropyl PG-dimonium chloride phosphate and methyl perfluorobutyl ether and methyl perfluoroisobutyl ether; and
a solvent.

19. The formulation of claim 18, wherein the solvent comprises water.

20. The formulation of claim 18, comprising:
1 to 30 weight percent surfactant;
0.1 to 10 weight percent thickener;
2 to 30 weight percent of the component comprising polyhydroxystearic acid and isononyl isononanoate and ethylhexyl isononanoate and sodium cocamidopropyl PG-dimonium chloride phosphate and methyl perfluorobutyl ether and methyl perfluoroisobutyl ether; and
10 to 90 weight percent of the solvent, the solvent comprising water.
